(19) United States
(12) Patent Application Publication
(10) Pub. No.: US 2025/0238924 A1
(43) Pub. Date: Jul. 24, 2025

(54) RADIOGRAPHIC IMAGE ANALYSIS APPARATUS AND NON-TRANSITORY COMPUTER-READABLE RECORDING MEDIUM STORING RADIOGRAPHIC IMAGE ANALYSIS PROGRAM

(71) Applicant: Konica Minolta, Inc., Tokyo (JP)

(72) Inventors: Takuya YAMAMURA, Tokyo (JP); Naoki HAYASHI, Tokyo (JP)

(21) Appl. No.: 19/028,025

(22) Filed: Jan. 17, 2025

(30) Foreign Application Priority Data
Jan. 18, 2024 (JP) 2024-006078

(51) Int. Cl. G06T 7/00; G16H 30/40

(52) U.S. Cl. CPC G06T 7/0012; G16H 30/40; G06T 2207/10124

(57) ABSTRACT

A radiographic image analysis apparatus comprising: a communicator that acquires from an image-capturing apparatus a radiographic image captured by irradiating a subject with radiation, and transmits the radiographic image acquired to a medical image management system; and one or more hardware processors, wherein the one or more hardware processors: analyze the radiographic image before transmission to the medical image management system by the communicator, determine whether an analysis result analyzed is appropriate, and present information about the radiographic image that is a reason for a determination to be inappropriate, when it is determined that the analysis result determined is inappropriate.

### CROSS REFERENCE TO RELATED APPLICATIONS

The present invention claims priority under 35 U.S.C. § 119 to Japanese Patent Application No. 2024-006078 filed on Jan. 18, 2024 the entire content of which is incorporated herein by reference.

### BACKGROUND

### Technological Field

The present invention relates to a radiographic image analysis apparatus and a non-transitory computer-readable recording medium storing a radiographic image analysis program.

### Description of Related Art

An analysis apparatus is known that acquires an analysis result of a disease or the like of a subject by irradiating the subject with radiation and analyzing a captured dynamic image. A dynamic image requires a longer image-capturing time than a still image, and therefore in a case where the subject moves during image-capturing, images of frames forming the dynamic image are blurred, and thus an analysis result may not be obtained.

Therefore, in the analysis apparatus disclosed in Japanese Unexamined Patent Publication No. 2018-175321, a dynamic image is pre-analyzed to immediately determine whether a sufficient analysis result can be obtained from the dynamic image, and when it is determined that the amount of information is insufficient, re-image-capturing is prompted.

Incidentally, the invention disclosed in Japanese Unexamined Patent Publication No. 2018-175321 determines whether analysis of a dynamic image is possible, but does not determine whether an analysis result is appropriate. Therefore, even if the analysis result is inappropriate, the analysis can be made, determining that the image-capturing processing has been completed so as to perform the next processing, for example, automatic transmission to a medical image management system (Picture Archiving and Communication System: PACS) or the like. In this case, for example, when the analysis result is determined to be inappropriate at the PACS, a dynamic image may be captured again, but since it takes time until a response is received from the PACS, the patient (subject) often returns home, which also causes time and effort (cost) such as a return visit of the patient or a re-inspection reservation.

The case where the analysis result is determined to be inappropriate does not mean that the state of the disease of the subject is diagnosed as abnormal, but means that the analysis result is determined not to have sufficient quality to enable reliable diagnosis of the state of the disease of the subject.

### SUMMARY

An object of the present invention is to provide a radiographic image analysis apparatus and a non-transitory computer-readable recording medium storing a radiographic image analysis program that can avoid determination that an analysis result is inappropriate on the medical image management system side.

In order to achieve at least one of the above-described objects, a radiographic image analysis apparatus reflecting one aspect of the present invention includes a communicator that acquires from an image-capturing apparatus a radiographic image captured by irradiating a subject with radiation, and transmits the radiographic image acquired to a medical image management system; and one or more hardware processors, in which the one or more hardware processors: analyze the radiographic image before transmission to the medical image management system by the communicator, determine whether an analysis result analyzed is appropriate, and present information about the radiographic image that is a reason for a determination to be inappropriate, when it is determined that the analysis result determined is inappropriate.

In order to achieve at least one of the abovementioned objects, a non-transitory computer-readable recording medium according to an aspect of the present invention stores a radiographic image analysis program for causing a computer provided in a radiographic image analysis apparatus, that acquires from an image-capturing apparatus a radiographic image captured by irradiating a subject with radiation, and transmits the radiographic image to a medical image management system, to perform: analyzing the radiographic image before transmission to the medical image management system; determining whether an analysis result of the radiographic image is appropriate; and presenting information about the radiographic image that is a reason for a determination to be inappropriate, when it is determined that the analysis result is inappropriate.

### BRIEF DESCRIPTION OF DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a diagram illustrating an example of a configuration of a radiographic image processing system according to an embodiment of the present invention.

FIG. 2 is a block diagram illustrating an example of a functional configuration of an image-capturing controller in the radiographic image-capturing apparatus making up the radiographic image processing system.

FIG. 3 is a block diagram illustrating an example of a functional configuration of the radiography control apparatus making up the radiographic image processing system.

FIG. 4 is a block diagram illustrating an example of a functional configuration of the radiographic image analysis apparatus making up the radiographic image processing system.

FIG. 5 is a flowchart illustrating a dynamic analysis performed by the radiographic image analysis apparatus illustrated in FIG. 1.

FIG. 6 is a diagram schematically illustrating a case where an analysis result is inappropriate, a determination process of determining whether or not the analysis result is appropriate, and an editing process in a case where the analysis result is inappropriate, for each mode of the dynamic analysis performed by the radiographic image analysis apparatus illustrated in FIG. 1.

FIG. 7 is a diagram schematically comparing the present invention and the prior art about information displayed on a radiographic image analysis apparatus.

### DETAILED DESCRIPTION OF EMBODIMENTS

Hereinafter, one or more embodiments of the present invention will be described with reference to the drawings. However, the scope of the invention is not limited to the disclosed embodiments.

Hereinafter, embodiments of the present invention will be described in detail with reference to the drawings.

### Configuration of Radiographic Image Processing System

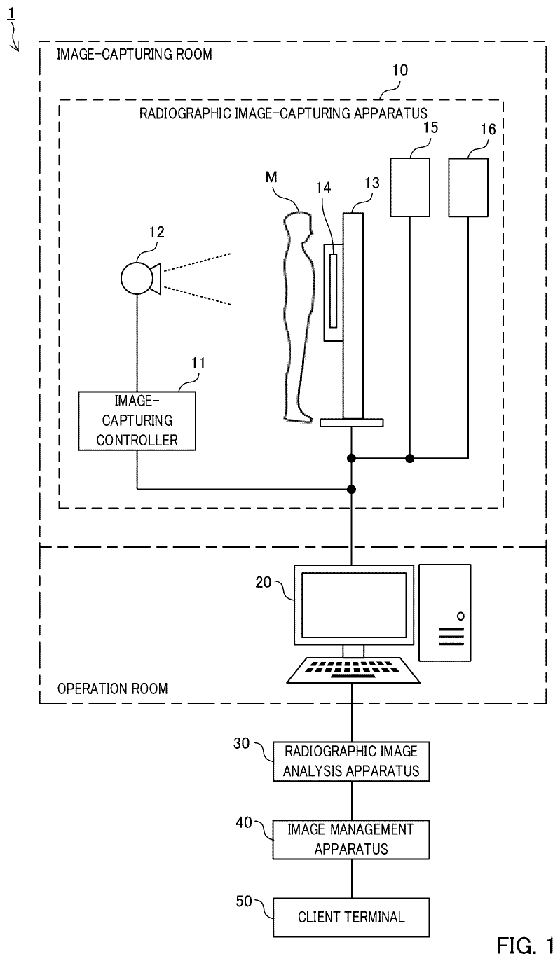

FIG. 1 is a diagram illustrating a radiographic image processing system 1 according to the present exemplary embodiment. The radiographic image processing system 1 includes a radiographic image-capturing apparatus 10, a radiography control apparatus (console apparatus) 20, a radiographic image analysis apparatus 30, an image management apparatus 40, and a client terminal 50.

In the example illustrated in FIG. 1, the radiographic image-capturing apparatus 10 is arranged in an image-capturing room, and the radiography control apparatus 20 is arranged in an operation room. The radiographic image-capturing apparatus 10, the radiography control apparatus 20, the radiographic image analysis apparatus 30, the image management apparatus 40, and the client terminal 50 are connected to each other via a communication network. As the communication network, for example, a communication network compliant with the DICOM (Digital Image and Communications in Medicine) standard or the like is used.

The radiographic image-capturing apparatus 10 captures a dynamic image that is a radiographic image under the control of the radiography control apparatus 20. The dynamic images generated by the radiographic image-capturing apparatus 10 are transmitted to the radiographic image analysis apparatus 30 via the radiography control apparatus 20. The radiographic image analysis apparatus 30 performs dynamic analysis on the dynamic image. The dynamic image and the result of the dynamic analysis are transmitted to and managed by an image management apparatus 40 (for example, PACS or the like) as a medical image management system. The dynamic image and the result of the dynamic analysis are transmitted to the client terminal 50 and viewed by a healthcare professional such as a physician.

Each of the radiographic image-capturing apparatus 10, the radiography control apparatus 20, and the radiographic image analysis apparatus 30 is a type of computer that includes a processor and a memory and realizes a predetermined function by reading, expanding, and executing a program stored in the memory.

### Radiographic Image-Capturing Apparatus 10

As illustrated in FIG. 1, the radiographic image-capturing apparatus 10 includes an image-capturing controller 11, a radiation emitter 12, an image-capturing table 13, a radiation detector 14, a display 15, and a sound outputter 16.

The image-capturing controller 11 acquires setting information regarding the setting of the radiation dynamic image capturing from the radiography control apparatus 20. The image-capturing controller 11 sets image-capturing conditions for performing dynamic image capturing based on the setting information, controls the radiation emitter 12 based on the image-capturing conditions to irradiate the subject M (for example, a patient) with radiation, and performs image-capturing. The image-capturing controller 11 includes a Central Processor (CPU), a Random Access Memory (RAM), a Read Only Memory (ROM), and the like.

The setting information is information on settings for performing dynamic image capturing on the subject M. The setting information includes, for example, at least one of a plurality of types of dynamic analysis that can be performed on the dynamic image by the radiographic image analysis apparatus 30. When a plurality of types of dynamic analyses are combined, the setting information may include information on the combination. The setting information is set by an operator of the radiographic image processing system 1, for example, an image-capturing technician, in the radiography control apparatus 20 to be described later.

The image-capturing conditions include, for example, various conditions such as a pulse rate, a pulse width, a pulse interval, the number of image-capturing frames per image-capturing, a dose per unit time of radiation irradiation, and a body state (breathing state or the like) of the subject M. The pulse rate is the number of times of radiation emission per second and matches the frame rate of image data. The pulse width is a radiation irradiation time per radiation irradiation. The pulse interval is a time from the start of one radiation irradiation to the start of the next radiation irradiation and coincides with a time interval (frame interval) between a plurality of image data. The image-capturing conditions may be automatically determined by the image-capturing controller 11 of the radiographic image-capturing apparatus 10 based on the setting information.

The radiation emitter 12 is disposed at a position facing the radiation detector 14 fixed to the image-capturing table 13. The radiation emitter 12 emits radiation (X-rays) under the control of the image-capturing controller 11.

The radiation detector 14 is configured by a solid-state image sensor such as an FPD (Flat Panel Detector). The radiation detector 14 includes a substrate on which a plurality of detection elements (pixels) that detect the radiation emitted from the radiation emitter 12 in accordance with the intensity of the radiation, convert the detected radiation into an electric signal, and accumulate the electric signal are arranged in a matrix. Each pixel of the substrate is configured to include a switcher such as a Thin Film Transistor (TFT), for example.

The radiation detector 14 controls the switcher of each pixel based on the image reading condition input from the radiography control apparatus 20 to read the electric signal accumulated in each pixel, and outputs intensity information of each pixel to an image generator 113. The image reading condition is, for example, a frame rate, a frame interval, a pixel size, an image size (matrix size), or the like. The frame rate is the number of frame images acquired per second and matches the pulse rate. The frame interval is a time period from the start of one operation of acquiring image data to the start of the operation of acquiring the next frame image and corresponds to the pulse interval.

The image-capturing controller 11 and the radiation detector 14 are connected to each other, and exchange synchronization signals with each other so as to synchronize the radiation irradiation operation and the image reading operation.

In this manner, the radiographic image-capturing apparatus 10 performs dynamic image capturing of a radiographic image by the radiation emitter 12 emitting radiation and the radiation detector 14 generating image data based on the intensity of the emitted radiation under the control of the image-capturing controller 11.

The display 15 and the sound outputter 16 give an instruction regarding a posture or a body state (a breathing state or the like) to be taken to the subject M when performing dynamic image capturing of the subject M. The display 15 is a display apparatus such as a Cathode Ray Tube (CRT), a Liquid Crystal Display (LCD), or an organic Electro Luminescence (EL) display, for example. The sound outputter 16 is, for example, a sound output apparatus such as a speaker. Each of the display 15 and the sound outputter 16 may provide an instruction having the same content to the subject M, or only one of them may provide an instruction.

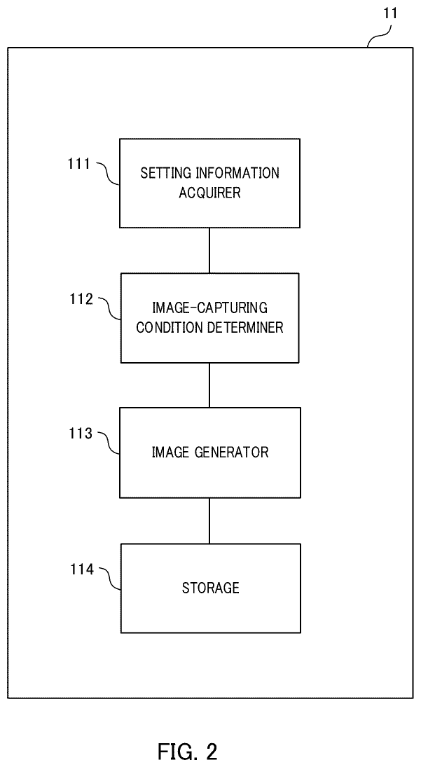

FIG. 2 is a block diagram illustrating an example of a functional configuration of the image-capturing controller 11 in the radiographic image-capturing apparatus 10 making up the radiographic image processing system 1. The image-capturing controller 11 includes a setting information acquirer 111, an image-capturing condition determiner 112, the image generator 113, and a storage 114.

The setting information acquirer 111 acquires setting information from the radiography control apparatus 20.

The image-capturing condition determiner 112 determines image-capturing conditions for performing dynamic image capturing of the subject M, based on the setting information. Information indicating a correspondence relationship between a plurality of types of dynamic analysis and image-capturing conditions suitable for the respective dynamic analysis is stored in the storage 114 in advance. Information indicating a correspondence relationship between a combination of a plurality of types of dynamic analysis and image-capturing conditions suitable for the combination is also stored in the storage 114 in advance. The image-capturing condition determiner 112 may determine the image-capturing condition by reading the information indicating the correspondence relationship from the storage 114 for the dynamic analysis indicated by the setting information or the combination of the plurality of types of dynamic analysis and collating the information with the setting information.

Note that, for example, in the case of screening, emergency, or the like, the dynamic analysis serving as the setting information may not be set. In such a case, the image-capturing condition determiner 112 determines the image-capturing condition by prompting the user to select at least one image-capturing condition from among a plurality of predefined image-capturing conditions. Further, the image-capturing condition determiner 112 allows the user to select an examination order and determines image-capturing conditions on the basis of the selected examination order. In this manner, in a case where the dynamic analysis cannot be set before the dynamic image capturing, the dynamic analysis is set after the dynamic image capturing under the image-capturing condition selected by the user, and the dynamic analysis or the like (for example, the analysis in an analyzer 312 or the determination in a determiner 313 to be described later) in the radiographic image analysis apparatus 30 to be described later is executed.

The image generator 113 executes dynamic image capturing of the subject M on the basis of the determined image-capturing conditions and generates a plurality of frames of radiographic images. More specifically, the image generator 113 controls the operations of the radiation emitter 12 and the radiation detector 14 based on the image-capturing conditions, and generates image data by acquiring, for each pixel, intensity information relating to the intensity of radiation transmitted through the subject from the radiation detector 14.

As described above, the storage 114 stores, in advance, information indicating a correspondence relationship between a plurality of types of dynamic analyses and image-capturing conditions suitable for the respective dynamic analyses, information indicating a correspondence relationship between combinations of the plurality of types of dynamic analyses and image-capturing conditions suitable for the combinations, and the like.

### Radiography Control Apparatus 20

The radiography control apparatus 20 is, for example, a computer such as a Personal Computer (PC) or a workstation. The radiography control apparatus 20 may be a desktop computer as in the example illustrated in FIG. 1 or may be a portable computer such as a notebook computer or a tablet computer.

The radiography control apparatus 20 controls dynamic image capturing of the radiographic image-capturing apparatus 10 by receiving inspection order information from an RIS (Radiology Information System; not illustrated in the drawing) or the like and transmitting the inspection order information to the radiographic image-capturing apparatus 10. The inspection order information includes various kinds of information about dynamic image capturing to be executed next, such as patient information, examination information, and data attributes. The examination information includes information such as examination IDs, examination target sites (for example, the thorax, particularly the lungs or the heart), and types of analysis (for example, ventilation analysis, pulmonary blood flow analysis, measurement of maximal voluntary ventilation, and the like). The inspection order information is generated, for example, when a doctor or the like requests the radiographic image processing system 1 to perform dynamic image capturing of the subject M.

In addition, the radiography control apparatus 20 generates setting information indicating at least one dynamic analysis among a plurality of kinds of dynamic analyses executable by the radiographic image analysis apparatus 30 on the basis of the input of the operator. In the case of combining a plurality of kinds of dynamic analysis, the radiography control apparatus 20 generates setting information indicating the combination of the plurality of kinds of dynamic analysis. The operator recognizes which dynamic analysis to combine among the plurality of types of dynamic analysis by referring to, for example, the content of the inspection order information and performs an input operation for generating the setting information on the basis of the recognition. Alternatively, the operator may recognize which dynamic analyses are to be combined, on the basis of information transmitted from a doctor or the like by another method.

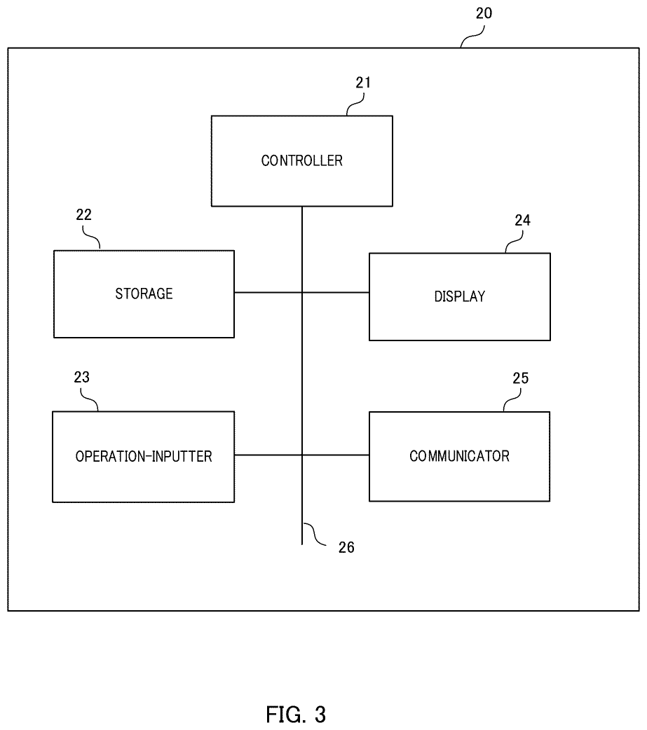

FIG. 3 is a block diagram illustrating an example of a functional configuration of the radiography control apparatus 20 making up the radiographic image processing system 1. The radiography control apparatus 20 includes a controller 21, a storage 22, an operation-inputter 23, a display 24, and a communicator 25. The components of the radiography control apparatus 20 are connected to each other via a bus 26.

The radiography control apparatus 20 outputs, to the radiographic image-capturing apparatus 10, setting conditions set by an operator or the like and inspection order information acquired from the RIS or the like in advance, and controls image-capturing processing by the radiographic image-capturing apparatus 10. The radiography control apparatus 20 may display the dynamic image generated by the radiographic image-capturing apparatus 10, for example, for the operator to check.

The controller 21 includes a CPU, a RAM, and the like. In the controller 21, in response to an operation of the operation-inputter 23, the CPU reads a system program and various processing programs stored in the storage 22, develops them in the RAM, and controls the operation of each part of the radiography control apparatus 20 based on the developed program.

The storage 22 is configured by a nonvolatile semiconductor memory, a hard disk, or the like. The storage 22 stores various programs executed by the controller 21, parameters necessary for execution of processing by the programs, or data such as processing results. The various programs are stored in the form of readable program codes, and the controller 21 sequentially executes operations according to the program codes.

Further, the storage 22 stores image reading conditions for performing dynamic image capturing. Further, the storage 22 stores inspection order information transmitted from the RIS or the like. When the radiography control apparatus 20 controls the dynamic image capturing of the radiographic image-capturing apparatus 10, it reads out the image reading conditions and the inspection order information corresponding to the subject M from the storage 22 and transmits the readout information.

The operation-inputter 23 is an operation apparatus such as a keyboard including cursor keys, number input keys, and various function keys, a pointing apparatus such as a mouse or a trackball, and a touch panel. The operation-inputter 23 generates an instruction signal based on an input of an operator, and outputs the instruction signal to the controller 21.

The display 24 is constituted by a display apparatus such as a CRT, a liquid crystal display, or an organic EL display. The display 24 displays an input instruction from the operation-inputter 23, image data generated by the radiographic image-capturing apparatus 10, and the like in accordance with an instruction of a display signal input from the controller 21.

The communicator 25 transmits and receives data to and from the radiographic image-capturing apparatus 10, the radiographic image analysis apparatus 30, the RIS, and the like.

### Radiographic Image Analysis Apparatus 30

The radiographic image analysis apparatus 30 is, for example, a computer such as a PC or a workstation. The radiographic image analysis apparatus 30 may be a desktop computer or a portable computer such as a notebook computer or a tablet computer. The radiographic image analysis program is stored in a non-transitory computer-readable recording medium, and the radiographic image analysis apparatus 30 reads the radiographic image analysis program from the recording medium.

The radiographic image analysis apparatus 30 performs a dynamic analysis on the dynamic image captured by the radiographic image-capturing apparatus 10 based on the setting information set in the radiography control apparatus 20. The radiographic image analysis apparatus 30 may execute a plurality of types of dynamic analyses in combination. However, in order to simplify the description, a case where one dynamic analysis is executed will be described below.

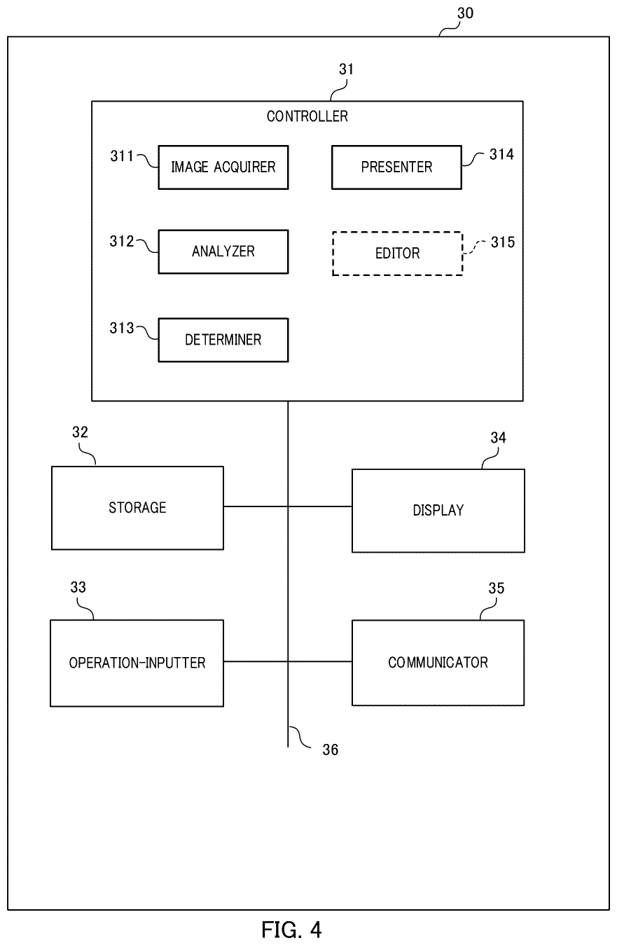

FIG. 4 is a block diagram illustrating an example of a functional configuration of the radiographic image analysis apparatus 30 making up the radiographic image processing system 1. The radiographic image analysis apparatus 30 includes a controller 31, a storage 32, an operation-inputter 33, a display 34, and a communicator 35. These components of the radiographic image analysis apparatus 30 are connected to each other by a bus 36.

The controller 31 includes a CPU, a RAM, and the like. In the controller 31, in response to an operation of the operation-inputter 33, the CPU reads a system program and various processing programs stored in the storage 32, develops them in the RAM, and executes operation control of each part of the radiographic image analysis apparatus 30, dynamic analysis, and the like on the basis of the developed programs.

The controller 31 includes an image acquirer 311, the analyzer 312, the determiner 313, and a presenter 314. The controller 31 may be composed of one or more hardware processors, and in that case, one or more hardware processors function as the image acquirer 311, the analyzer 312, the determiner 313, the presenter 314, and an editor 315 described later.

The image acquirer 311 acquires a dynamic image which is a plurality of frames of radiographic images generated by the radiographic image-capturing apparatus 10.

The analyzer 312 performs the dynamic analysis set in the setting information on the dynamic image acquired from the radiographic image-capturing apparatus 10, and acquires an analysis result. At this time, when the analyzer 312 cannot analyze the dynamic image (cannot acquire the analysis result), the analyzer 312 determines that the analysis is not possible.

The analyzer 312 has, for example, a blood flow analysis mode, a ventilation analysis mode, an adhesion analysis mode, a diaphragm movement amount analysis mode, an orthopedic-related measurement mode, and the like as types of dynamic analysis (analysis modes in the present invention). Each mode will be briefly described below.

The blood flow analysis mode is a mode in which a signal change in the lung field synchronized with the heartbeat is visualized.

The ventilation analysis mode is a mode in which a signal change in a time direction in a specific time-frequency band is extracted and lung tissue behavior during breathing is visualized.

The adhesion analysis mode is a mode for visualizing the degree of adhesion of tissues.

The diaphragm movement amount analysis mode is a mode for tracking the up/down movement of the diaphragm associated with breathing.

The orthopedic-related measurement mode is, for example, a mode in which a change in the position of a specified bone in the four limbs or the like is measured and the trajectory of the movement is displayed.

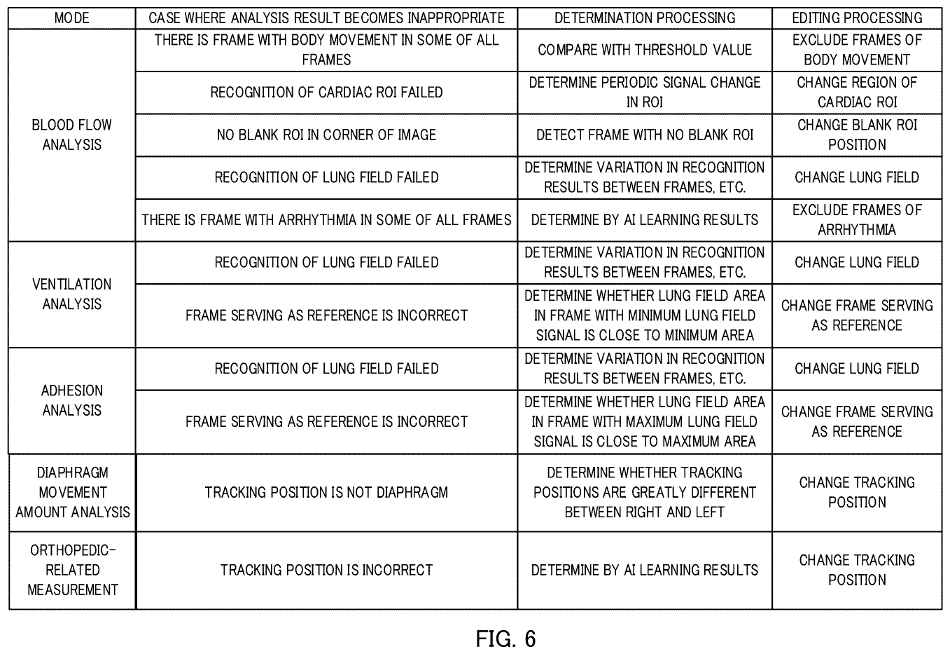

When the analysis result can be acquired by the analyzer 312, the determiner 313 determines whether or not the acquired analysis result is appropriate. The determination of whether or not the analysis result is appropriate will be specifically described with reference to FIG. 6 described later.

When the analyzer 312 can analyze the dynamic image and the determiner 313 determines that the analysis result is appropriate, the presenter 314 displays the analysis result together with the dynamic image on the display 34 and presents the analysis result to the operator. When the analyzer 312 cannot analyze the dynamic image, the presenter 314 displays an error on the display 34 and presents the error to the operator. Further, if the determiner 313 determines that the analysis result is inappropriate, the presenter 314 displays, on the display 34, information on the dynamic image that is the cause of the determination and presents it to the operator. The information on the dynamic image that is the cause determined to be inappropriate will also be specifically described with reference to FIG. 6 described later.

The operator manually edits the dynamic image based on the information presented by the presenter 314 (information about the dynamic image that is the cause of the determination that the dynamic image is inappropriate). The controller 31 may further include the editor 315 to edit the dynamic image. In this case, the editor 315 automatically or semi-automatically edits the dynamic image based on the information presented by the presenter 314.

The storage 32 is configured by a nonvolatile semiconductor memory, a hard disk, or the like. The storage 32 stores various programs executed by the controller 31, parameters necessary for execution of processing by the programs, or data such as processing results. The various programs are stored in the form of readable program codes, and the controller 31 sequentially executes operations in accordance with the program codes.

The storage 32 also stores patient information and examination information relating to each dynamic image generated by the radiographic image-capturing apparatus 10, and list information indicating the status (e.g., progress status such as receiving, dynamic analysis, and analysis completed). Further, analysis results are stored in the storage 32 in association with the dynamic images.

The operation-inputter 33 is an operation apparatus such as a keyboard including cursor keys, number input keys, and various function keys, a pointing apparatus such as a mouse or a trackball, and a touch panel. The operation-inputter 33 generates an instruction signal based on an input by the operator, and outputs the instruction signal to the controller 31. The operation-inputter 33 may include a touch panel on the display screen of the display 34, and in this case, outputs an instruction signal input through the touch panel to the controller 31.

The display 34 is constituted by a display apparatus such as a CRT, a liquid crystal display, or an organic EL display. The display 34 displays an input instruction from the operation-inputter 33, image data generated by the radiographic image-capturing apparatus 10, and the like according to an instruction of a display signal input from the controller 31.

The communicator 35 transmits and receives data to and from the radiography control apparatus 20, the image management apparatus 40, and the like.

The analysis processing of the dynamic image and the presentation processing of the information relating to the dynamic image which is the cause determined to be inappropriate in the radiographic image analysis apparatus 30 having the above-described configuration will be described below.

### Dynamic Analysis

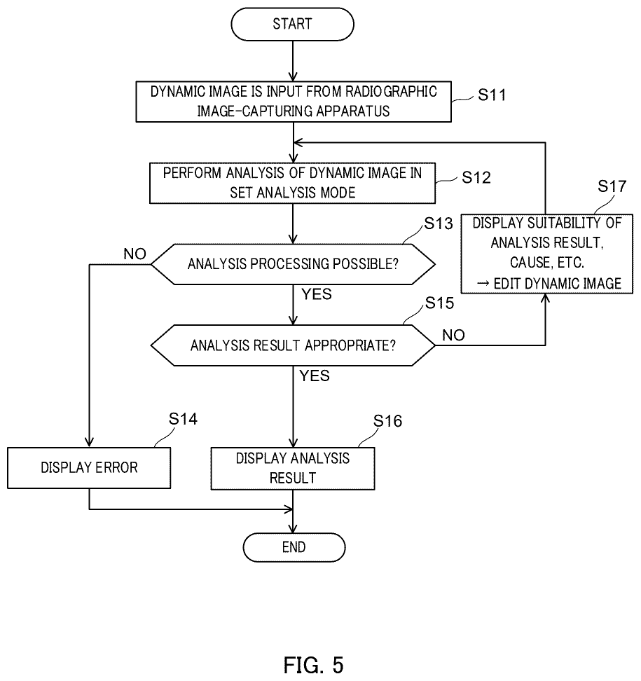

FIG. 5 is a flowchart illustrating the dynamic analysis performed by the radiographic image analysis apparatus 30. The dynamic analysis will be described with reference to FIG. 5 and also FIGS. 1 to FIG. 4

### Step S11

When a dynamic image is captured by irradiating the subject M with radiation by the radiographic image-capturing apparatus 10 and the radiography control apparatus 20, the dynamic image is input to the radiographic image analysis apparatus 30 from the radiographic image-capturing apparatus 10 and the radiography control apparatus 20.

### Step S12

In the radiographic image analysis apparatus 30, the analyzer 312 performs the set dynamic analysis on the dynamic image. As the dynamic analysis, for example, the above-described blood flow analysis mode, ventilation analysis mode, adhesion analysis mode, diaphragm movement amount analysis mode, orthopedic-related measurement mode and the like are set and executed.

### Step S13

The analyzer 312 executes the set dynamic analysis on the dynamic image and proceeds to step S15 when the analysis processing of the dynamic image can be performed (YES), and proceeds to step S14 when the analysis processing of the dynamic image cannot be performed (NO).

### Step S14

If the analyzer 312 cannot analyze the dynamic image (NO in step S13), the presenter 314 displays an error on the display 34, presents the error to the user, and ends the series of processes.

### Step S15

In a case where the analysis processing of the dynamic image can be performed by the analyzer 312 (YES in step S13), that is, in a case where the analysis result can be acquired by the analyzer 312, the determiner 313 determines whether or not the analysis result is appropriate. If the analysis result is appropriate (YES), the process proceeds to step S16. If the analysis result is inappropriate (NO), the process proceeds to step S17. The determination of whether or not the analysis result is appropriate will be specifically described with reference to FIG. 6 described later.

### Step S16

If the determiner 313 determines that the analysis result is appropriate (YES in step S15), the presenter 314 displays the analysis result together with the dynamic image on the display 34 to present them to the user and ends the series of processing.

### Step S17

When the determiner 313 determines that the analysis result is inappropriate (NO in step S15), the presenter 314 displays, on the display 34, information on the dynamic image that is the cause of the determination that the analysis result is inappropriate and presents the information to the user. The information on the dynamic image that is the cause of the determination as inappropriate will be specifically described with reference to FIG. 6 described later. Then, the operator manually edits the dynamic image on the basis of the presented information. Alternatively, the editor 315 automatically or semi-automatically edits the dynamic image on the basis of the presented information.

After the editing of the dynamic images in step S17, Returning to step S12, the edited dynamic image is re-analyzed. If the editing of the dynamic image is appropriate, the result of the re-analysis is determined to be appropriate, and a series of processing ends via steps S13 to S16. Even if editing of the dynamic image is not appropriate, in that case, through steps S13 to S15, the result of re-analysis is determined to be inappropriate, and the processing in step S17 is performed again, so that re-editing of the dynamic image is performed, and more appropriate editing is possible.

Here, a case in which an analysis result is determined to be inappropriate in each mode of the dynamic analysis, and determination processing and editing processing therefor will be described with reference to FIG. 6.

### Blood Flow Analysis Mode

When the dynamic analysis is in the blood flow analysis mode, the case where the analysis result is determined to be inappropriate, the determination process, and the editing process include, for example, the case illustrated in FIG. 6, the determination items, and the editing items.

In the blood flow analysis mode, for example, an analysis result is determined to be inappropriate in a case where there are frames of body movement in some of all the frames of the dynamic image. As a specific example, when the subject M is made to hold his/her breath and a dynamic image is captured, and the breath holding is not performed and there is body movement, it is determined that the analysis result is inappropriate although the analysis can be performed.

In this case, as a determination process, the determiner 313 compares a threshold value for determining a body movement with a movement amount of a predetermined structure for each frame, and determines that the analysis result is inappropriate when there is a frame in which the movement amount is equal to or greater than the threshold value. As the presentation processing, for example, the presenter 314 presents, to the operator, a frame in which the amount of movement of the predetermined structure captured in the dynamic image is equal to or more than a threshold, that is, a frame with a body movement.

Then, as an editing process, the operator or the editor 315 specifies a frame in which the movement amount of a predetermined structure captured in the dynamic image is equal to or greater than a threshold value, that is, a frame with a body movement, and deletes the frame from all the frames of the dynamic image.

In this manner, since a frame with a body movement is deleted from among all the frames of the dynamic image, it is possible to acquire an appropriate analysis result.

Note that, for example, when the editor 315 has a body movement correction function or the like for correcting a body movement, the operator or the editor 315 corrects the body movement in a frame with the body movement by the body movement correction function as the editing processing.

In this manner, since the body movement is corrected in the frame with the body movement, it is possible to acquire an appropriate analysis result.

Regarding the above-described suitability of an analysis result, the suitability of an analysis result may be determined on the basis of whether or not body movement can be corrected in a frame with the body movement, in consideration of the presence or absence, and a combination, of the above-described body movement correction function and other image processing functions (e.g., a distortion correction function based on warping processing, etc).

In addition, the above-described suitability of the analysis result is not limited to the thresholds for determining the body movement for each frame, and the number of frames in which the body movement occurs among all the frames of the dynamic image or the occurrence rate may be used as thresholds for the determination. In the case of the number of frames, for example, when the number of frames with a body movement is 11 to 30 in the dynamic images of 100 frames, it is determined that the analysis result is inappropriate. Also in this case, an appropriate analysis result can be acquired by deleting a frame with a body movement from all the frames of the dynamic image. Note that when the number of frames with body movement is 31 or more, it may be determined that analysis is not possible.

The suitability of the analysis result described above is not limited to the thresholds, and may be determined using the presence or absence of a body movement within the breathing cycle in the dynamic image. In this case, for example, if a body movement occurs in one breathing cycle in a dynamic image having two breathing cycles, it is determined that the analysis result is inappropriate. Also in this case, it is possible to acquire an appropriate analysis result by deleting one breathing cycle frame with a body movement from the frames of two breathing cycles. When the body movement occurs in both of the two breathing cycles, it may be determined that the analysis is not possible.

In addition, in a case where recognition of cardiac ROI (Region of Interest) fails in the blood flow analysis mode, the analysis result is determined to be inappropriate. As a specific example, in the case of a patient having a large body thickness such as an obese patient, recognition of cardiac ROI may fail, and in this case, even if analysis can be performed, the analysis result is determined to be inappropriate.

In this case, as determination processing, the determiner 313 checks a periodic signal change in the recognized ROI and determines that the analysis result is inappropriate in a case where there is no periodic signal change. As the presentation processing, the presenter 314 presents, to the operator, for example, that there is no periodic signal change in the recognized ROI.

Then, as an editing process, the operator or the editor 315 changes the ROI in the dynamic image to a region in which a periodic signal change can be confirmed, that is, a region including the heart.

In this manner, the ROI in the dynamic image is changed to the region including the heart, so that an appropriate analysis result can be acquired.

In addition, in the blood flow analysis mode, in a case where there is no blank ROI (air region) in a corner part of the image, for example, in an upper left region of the image, there is no region to be used as a reference for analysis, and even if the analysis can be performed, the analysis result is determined to be inappropriate.

In this case, as determination processing, the determiner 313 determines that the analysis result is inappropriate in a case where there is a frame in which a blank ROI is absent in a corner portion of the image (e.g., an upper left region of the image). As the presentation processing, the presenter 314 presents, to the operator, for example, a frame in which no blank ROI is present in a corner portion of the image.

Then, the operator or the editor 315 changes the blank ROI in the dynamic image to an appropriate region (air region) as an editing process. Not limited to the region change of the blank ROI, the gradation of the blank ROI may be corrected, or a frame in which the blank ROI is absent may be deleted.

In this manner, since the blank ROI in the dynamic image is changed to an appropriate region, an appropriate analysis result can be acquired.

Further, in the blood flow analysis mode, when the recognition of the lung field fails, the mask image of the lung field is not appropriately set, and the analysis result is determined to be inappropriate. As a specific example, in a case of a patient or the like whose state of an ICU or the like is bad, recognition of a lung field may fail, and in this case, even if analysis can be performed, an analysis result is determined to be inappropriate.

In this case, as the determination processing, the determiner 313 checks a variation in the recognition results of the lung field between frames, and determines that the analysis result is inappropriate when the variation is large. In addition, since the position of the diaphragm does not greatly change between the left and right, the determiner 313 may determine that the analysis result is inappropriate in a case where there is a large difference in the position of the lung base in the recognition results of the left and right lung fields as the determination process. As the determination processing, the determiner 313 may determine that the analysis result is inappropriate in a case where a level difference is large in the profile in the vertical direction of the mask image (in a case where the mask image protrudes from the region of the lung field). As the presentation processing, the presenter 314 presents, to the operator, for example, an inter-frame recognition result of the lung field, recognition results of the right and left lung fields, and vertical profiles of mask images.

Then, the operator or the editor 315 changes the lung field in the dynamic image to an appropriate region as an editing process. For example, the lung field is changed to an appropriate region with reference to the inter-frame lung field recognition result, the right and left lung field recognition results, and the vertical profile of the mask image.

In this manner, since the lung field in the dynamic image is changed to an appropriate region, an appropriate analysis result can be acquired.

Further, in the blood flow analysis mode, when there is an arrhythmia frame in some of all the frames of the dynamic images, it is determined that the analysis result is inappropriate.

In this case, as a determination process, the determiner 313 determines whether or not there is a frame of arrhythmia based on a learning result obtained by learning the frame of arrhythmia by Artificial Intelligence (AI). When there is a frame of arrhythmia, the determiner 313 determines that the analysis result is inappropriate. As the presentation processing, the presenter 314 presents, for example, a frame of arrhythmia to the operator.

Then, as an editing process, the operator or the editor 315 specifies a frame in which arrhythmia has occurred and deletes the frame from all the frames of the dynamic image.

Since a frame in which arrhythmia has occurred is deleted from among all the frames of the dynamic image in this manner, an appropriate analysis result can be acquired.

### Ventilation Analysis Mode

In a case where the dynamic analysis is in the ventilation analysis mode, the case where the analysis result is determined to be inappropriate, the determination process therefor, and the editing process therefor include, for example, the case, the determination items, and the editing items illustrated in FIG. 6.

In the ventilation analysis mode, similarly to the above-described blood flow analysis mode, in a case where the recognition of the lung field fails, the mask image of the lung field is not appropriately set, and the analysis result is determined to be inappropriate. In this case, it is possible to acquire an appropriate analysis result by performing the same processing as the determination processing, the presentation processing, and the editing processing in the blood flow analysis mode described above.

In addition, in the ventilation analysis mode, in a case where the frame serving as a reference is not correct, that is, in a case where the frame serving as a reference is not a frame at the time of maximum exhalation, it is determined that the analysis result is inappropriate. As a specific example, in the case of a patient with pneumothorax or the like, there is a possibility that the lung field is erroneously recognized, and in this case, there is a possibility that the frame used as a reference is not correct.

In this case, as a determination process, the determiner 313 determines whether the area of the lung field in a reference frame (a frame in which the signal value in the lung field is the smallest) is close to the area of the lung field in a frame in which the lung field has the smallest area. Then, when the areas of the lung fields are greatly separated from each other, the determiner 313 determines that the analysis result is inappropriate because the reference frame is not the frame corresponding to the maximum exhalation. As the presentation processing, the presenter 314 presents, to the operator, for example, the area of the lung field in a frame serving as a reference and the area of the lung field in a frame in which the lung field has the smallest area.

Then, as an editing process, the operator or the editor 315 refers to the area of the lung field of each frame, and changes the reference frame to the frame at the maximum exhalation.

In this manner, since the reference frame is changed to the frame at the maximum exhalation, it is possible to acquire an appropriate analysis result.

### Adhesion Analysis Mode

In a case where the dynamic analysis is in the adhesion analysis mode, the case where the analysis result is determined to be inappropriate, and the determination process and the editing process thereof include, for example, the case, the determination items, and the editing items illustrated in FIG. 6.

Also in the adhesion analysis mode, similarly to the blood flow analysis mode described above, when the recognition of the lung field fails, the mask image of the lung field is not appropriately set, and the analysis result is determined to be inappropriate. In this case, it is possible to acquire an appropriate analysis result by performing the same processing as the determination processing, the presentation processing, and the editing processing in the blood flow analysis mode described above.

In the ventilation analysis mode, when the reference frame is not correct, that is, when the reference frame is not a frame at the end of inspiration, the analysis result is determined to be inappropriate.

In this case, as the determination processing, the determiner 313 determines whether the area of the lung field in the reference frame (the frame in which the signal value in the lung field is the largest) is close to the area of the lung field in the frame in which the lung field has the largest area. Next, when the areas of the lung fields are greatly separated from each other, the determiner 313 determines that the analysis result is inappropriate because the frame used as a reference is not a frame at the end of inspiration. As the presentation processing, the presenter 314 presents, to the operator, for example, the area of the lung field in a frame serving as a reference and the area of the lung field in a frame in which the lung field has the largest area.

Then, as an editing process, the operator or the editor 315 refers to the area of the lung field of each frame and changes the reference frame to a frame at the end of inspiration.

In this manner, since the reference frame is changed to the frame at the end of inspiration, an appropriate analysis result can be acquired.

### Diaphragm Movement Amount Analysis Mode

In a case where the dynamic analysis is in the diaphragm movement amount analysis mode, the case where the analysis result is determined to be inappropriate, and the determination processing and the editing processing thereof include, for example, the case, the determination items, and the editing items illustrated in FIG. 6.

In the diaphragm movement amount analysis mode, for example, when the tracking position is not on the diaphragm, the analysis result is determined to be inappropriate. As a specific example, in the case of a patient with a bad lung condition, there is a possibility that the diaphragm cannot be correctly recognized, and if the tracking position is not the diaphragm, even if the analysis can be performed, the analysis result is determined to be inappropriate.

Since the position of the diaphragm does not greatly change between the right and left, as the determination processing, the determiner 313 checks whether the tracking positions greatly differ between the right and left, and if so, determines that the analysis result is inappropriate. As the presentation processing, the presenter 314 presents, for example, right and left tracking positions to the operator.

Next, the operator or the editor 315 changes the tracking position with reference to the right and left tracking positions as editing processing.

Since the tracking position is changed in this manner, an appropriate analysis result can be acquired.

### Orthopedic-Related Measurement Mode

In a case where the dynamic analysis is in the orthopedic-related measurement mode, the case where the analysis result is determined to be inappropriate, and the determination process and the editing process thereof include, for example, the case, the determination items, and the editing items illustrated in FIG. 6.

In the orthopedic-related measurement mode, for example, when the initial position of the tracking is not correct, the analysis result is determined to be inappropriate. As a specific example, the four corners of the bones of the cervical spine are set as the initial positions of tracking. However, depending on the direction of image-capturing or the condition of the patient, the initial position of tracking (e.g., the initial position of the cervical spine) may be incorrect if one of the bones of the cervical spine overlaps the shoulder on the image. Further, even for a patient in a poor condition due to compression fracture or the like, the bones on the screen may overlap each other and the initial position of the tracking may become incorrect. In such a case, even if the analysis can be performed, the analysis result is determined to be inappropriate.

In this case, as determination processing, the determiner 313 determines, based on a learning result obtained by learning the images of the bones of interest by AI, whether the bones overlap each other, and determines that the analysis result is inappropriate in a case where the bones overlap each other. As the presentation processing, the presenter 314 presents, to the operator, the positions of the bones in an overlapping manner, for example.

Then, as an editing process, the operator or the editor 315 changes the initial position of the tracking to an appropriate position as the initial position of the tracking, or changes the frame for setting the initial position of the tracking to set the initial position of the tracking. For example, in the case of the cervical spine, the initial position of tracking is set by changing to a frame in which the four corners of the bone can be seen.

In this manner, since the initial position of the tracking is changed or the initial position of the tracking is set in an appropriate frame, an appropriate analysis result can be acquired.

The suitability of the analysis result described in each mode of the dynamic analysis is not limited to the above-described determination, and may be determined by, for example, an indicator such as accuracy.

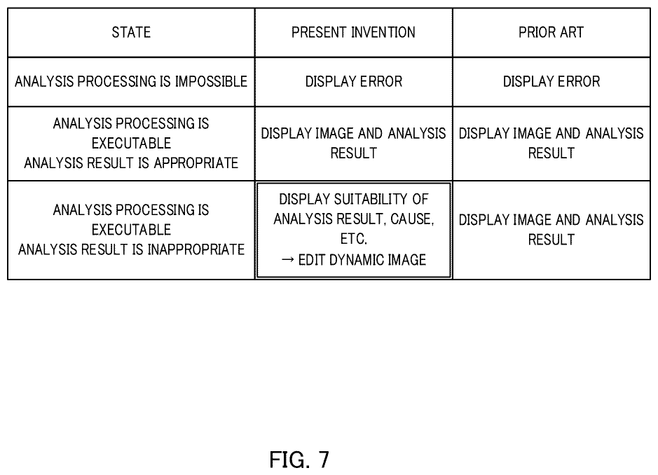

Here, FIG. 7 is a diagram schematically comparing the present invention and the prior art with respect to information displayed by the radiographic image analysis apparatus 30.

As illustrated in FIG. 7, the conventional radiographic image analysis apparatus displays an error when the analysis process is not possible, and displays the image and the analysis result when the analysis process is possible and the analysis result is appropriate. Also, when the analysis processing is possible and the analysis result is inappropriate, the image and the analysis result are displayed.

As illustrated in FIG. 7, the radiographic image analysis apparatus 30 according to the present embodiment is the same as in the prior art in a case where the analysis process is not possible and in a case where the analysis process is possible and the analysis result is appropriate. On the other hand, in a case where the analysis process can be performed and the analysis result is inappropriate, the radiographic image analysis apparatus 30 displays the suitability of the analysis result or information regarding the dynamic image which is the cause of the determination that the analysis result is inappropriate. Further, the radiographic image analysis apparatus 30 may display the dynamic image so that the above-described editing can be performed.

As described above, when a dynamic image is analyzed by the radiographic image analysis apparatus 30, it is determined whether or not the dynamic image can be analyzed. Further, even when the dynamic image can be analyzed, it is determined whether or not the analysis result is appropriate according to each mode of the dynamic analysis as described above. Therefore, before the analysis result is transmitted to the image management apparatus 40 (for example, PACS or the like), the suitability and accuracy of the analysis result can be determined early on the side of the radiographic image analysis apparatus 30.

Then, when the analysis result is inappropriate, an appropriate analysis result may be acquired by editing the dynamic image, and therefore, as described above, information on the dynamic image that is the cause of the determination that the dynamic image is inappropriate is presented according to each mode of the dynamic analysis. Therefore, the operator or the editor 315 can appropriately edit the dynamic image based on the information presented in accordance with each mode of the dynamic analysis and can acquire an appropriate analysis result. That is; by effectively using the captured dynamic image and appropriately editing the dynamic image, an appropriate analysis result can be acquired without re-image-capturing. As a result, it is possible to avoid the necessity of re-image-capturing of a dynamic image due to determination on the medical image management system (PACS) side that an analysis result is inappropriate, to reduce time and costs related to image-capturing and analysis, and to reduce re-exposure of the subject M to radiation.

### Modification Example

The example illustrated in FIG. 6 is for simply determining a case where analysis is possible, but an analysis result is inappropriate. The present invention is not limited thereto, and the suitability of the analysis result may be simply determined by the determination process illustrated in FIG. 6 without performing the analysis immediately after the dynamic image capturing. In this case, in the flowchart illustrated in FIG. 5, the suitability of the analysis result may be determined in step S15 without performing steps S12 to S14, S16, and S17 after step S11.

As described above, in a case where the suitability of the analysis result is determined by the determination processing illustrated in FIG. 6 without performing the analysis immediately after the dynamic image capturing, the user operation is unnecessary when the analysis result is appropriate, and the suitability of the analysis result can be determined earlier than in a case where the analysis is performed when the analysis result is inappropriate. Therefore, according to the determination content of the suitability of the analysis result, it is possible to make an early determination to perform re-image-capturing before returning the patient, or to wait for the patient to determine whether or not the dynamic image can be optimized by response or fine adjustment in the radiographic image analysis apparatus 30.

In the case where the simple determination is performed immediately after the dynamic image capturing, the simple determination may be performed in the radiography control apparatus 20 by cutting out the functions of the radiographic image analysis apparatus 30 as a program and providing the program to the radiography control apparatus 20. Alternatively, the determination result of the radiographic image analysis apparatus 30 may be externally output to a terminal, such as the radiography control apparatus 20, having a display function that allows the technician to check the determination result.

The above-described embodiments are merely examples for implementing the present invention, and the technical scope of the present invention should not be interpreted in a limited manner by these embodiments. That is, the present invention can be implemented in various forms without departing from the spirit or main features thereof.

Although embodiments of the present invention have been described and illustrated in detail, the disclosed embodiments are made for purpose of illustration and example only and not limitation. The scope of the present invention should be interpreted by terms of the appended claims.

What is claimed is:

1. A radiographic image analysis apparatus comprising:
a communicator that acquires from an image-capturing apparatus a radiographic image captured by irradiating a subject with radiation, and transmits the radiographic image acquired to a medical image management system; and
one or more hardware processors,
wherein the one or more hardware processors:
analyze the radiographic image before transmission to the medical image management system by the communicator,
determine whether an analysis result analyzed is appropriate, and
present information about the radiographic image that is a reason for a determination to be inappropriate, when it is determined that the analysis result determined is inappropriate.

2. The radiographic image analysis apparatus according to claim 1, wherein the one or more hardware processors:
edit the radiographic image based on the information,
perform re-analysis of the radiographic image edited, and
determine whether a re-analysis result obtained by the re-analysis is appropriate.

3. The radiographic image analysis apparatus according to claim 1,
wherein the radiographic image is a dynamic image, and
wherein the information is at least one of a specific frame making up the radiographic image, a specific region in the radiographic image, and a specific position in the radiographic image.

4. The radiographic image analysis apparatus according to claim 3, wherein the one or more hardware processors:
edit the radiographic image by performing at least one of deletion of the specific frame, change of the specific region, and change of the specific position in the radiographic image based on the information,
perform re-analysis of the radiographic image edited, and
determine whether a re-analysis result obtained by the re-analysis is appropriate.

5. The radiographic image analysis apparatus according to claim 1, wherein the one or more hardware processors include a plurality of analysis modes and at least one determination item corresponding to each of the plurality of analysis modes and detect a reason for a determination to be inappropriate based on the determination item.

6. A non-transitory computer-readable recording medium storing a radiographic image analysis program for causing a computer provided in a radiographic image analysis apparatus, that acquires from an image-capturing apparatus a radiographic image captured by irradiating a subject with radiation, and transmits the radiographic image to a medical image management system, to perform:
analyzing the radiographic image before transmission to the medical image management system;
determining whether an analysis result of the radiographic image is appropriate; and
presenting information about the radiographic image that is a reason for a determination to be inappropriate, when it is determined that the analysis result is inappropriate.
